(19) United States
(12) Patent Application Publication
(10) Pub. No.: US 2023/0168253 A1
(43) Pub. Date: Jun. 1, 2023

(54) SYSTEM AND METHOD FOR TESTING BIOLOGICAL SAMPLES

(71) Applicant: GRAPHENE-DX. INC., Boston, MA (US)

(72) Inventors: Tom RUBY, Boston, MA (US); Stephen PIZZO, Brookline, MA (US); Kyle BULLOCH, Rancho Santa Fe, CA (US); Namal NAWANA, Weston, MA (US)

(21) Appl. No.: 18/072,106

(22) Filed: Nov. 30, 2022

Related U.S. Application Data
(60) Provisional application No. 63/285,032, filed on Dec. 1, 2021.

(51) Int. Cl. G01N 33/68; G16B 30/00; G01N 33/493; G01N 1/38

(52) U.S. Cl. CPC G01N 33/68; G16B 30/00; G01N 33/493; G01N 1/38; G01N 2001/2893

(57) ABSTRACT

Systems and methods are provided for analyzing a test sample is provided. A test sampling apparatus includes a housing extending from a first end to a second end opposite the first end. The housing includes a chamber disposed at the first end. The chamber is configured to receive a test sample from a user. The test sampling apparatus includes a test sensor fluidically coupled to the chamber and disposed within an interior portion of the housing. The test sensor is configured to receive the test sample and generate information based thereon. The information includes data identifying a substance in the test sample. The test sampling apparatus also includes one or more processors disposed within an interior portion of the second end and in communication with the test sensor. The one or more processors receive the information from the test sensor and analyze the test sample based on the information.

### RELATED APPLICATIONS

This application claims priority to U.S. Provisional Patent Application No. 63/285,032, filed Dec. 1, 2021, entitled “Systems and Methods for Testing Biological Samples,” which is hereby incorporated by reference in their entirety.

### TECHNICAL FIELD

The present disclosure relates generally to systems and methods for testing biological test samples, and more particularly to a test sampling apparatus configured to collect and test biological samples using a test sensor.

### BACKGROUND

Biological samples provide important information about a variety of physiological conditions. For example, urine testing is a diagnostic technique that can be used to identify a variety of markers, such as markers of inflammatory, and renal tubular proteins, among others. Due to its ease of collection, urine testing can be employed for chronological assessment of such markers. As such, there is a need for easily accessible urine testing devices that identify such markers. Accordingly, there is a need for inexpensive and easy to use testing devices that collect and rapidly test biological samples

### SUMMARY

The present disclosure is directed to a system for collecting a biological sample and (optionally) analyzing the collected sample to determine whether a target analyte is present in the sample. In particular, the present disclosure describes systems and methods for conveniently collecting and testing a biological test sample collected from the user. For example, the biological test sample can include or consist of urine, which a user can easily test from home. The disclosed systems and methods identify one or more target analytes (e.g., a particular substance of the test sample under test, such as biological specimens) in the test sample, and present to the user an easily understandable representation of the test results.

In accordance with some embodiments, a test sampling apparatus includes a housing extending from a first end to a second end opposite the first end. The housing includes a chamber disposed at the first end. The chamber is configured to receive a test sample from a user. The test sampling apparatus includes a test sensor fluidically coupled to the chamber and disposed within an interior portion of the housing. The test sensor is configured to receive a portion of the test sample and generate information based thereon. The information can include data identifying one or more substances in the portion of the test sample. The test sampling apparatus further includes one or more processors disposed within an interior portion of the second end and in communication with the test sensor. The one or more processors are configured to receive the information from the test sensor and analyze the test sample based on the information. The test sampling apparatus also includes a cover configured to enclose the first end of the housing such that the chamber is covered. In some embodiments, test sampling apparatus further includes a display disposed on an external portion of the housing and responsive to the one or more processors. The display is configured to present a test analysis to the user.

In some embodiments, the cover is configured to slidably retract to expose the chamber. Alternatively, in some embodiments, the cover is a cap configured to couple to the first end of the housing. In some embodiments, test sampling apparatus includes an absorbent material layer disposed over the chamber. The absorbent material layer is configured to receive the test sample received from the user and store the test sample. In some embodiments, the absorbent material layer is configured to disperse the test sample to the chamber when the cap is coupled to the first end of the housing. In some embodiments, the cap includes a plurality of ribs for compressing the absorbent material layer

In some embodiments, the test sensor is disposed at the first end of the housing. In some embodiments, the test sensor is disposed at the second end of the housing, and the test sampling apparatus further includes a channel for transferring the test sample from the chamber to the test sensor. In some embodiments, the test sensor is integral with the one or more processors. In some embodiments, the test sensor is one of a FET-type device, a ChemFET-type device, EChemFET-type device, or electrochemical-type device. In some embodiments, the test sensor is an immuno-assay. In some embodiments, isothermal DNA amplification can be employed to amplify the DNA and hence facilitate genetic screening for biomarkers. In some embodiments, the test sensor can detect proteins or fragments of proteins. In some embodiments, the test sensor can detect different forms of nucleic acid (including but not limited to DNA, mRNA, micro-RNA, siRNA). In some embodiments, the test sensor can detect nucleic acid via amplification of specific nucleic acid sequence, or via detection of specific nucleic acid sequences, for instance through hybridization to an oligonucleotide or through a catalytically inactive CRISPR complex with a sgRNA having an oligonucleotide sequence that is complementary with a DNA sequence of interest.

In some embodiments, the test sampling apparatus includes a buffer liquid used to calibrate the test sensor. In some embodiments, the buffer liquid is stored within a foil seal, and the cover is configured to pierce the foil seal upon first actuation. In some embodiments, the test sensor is pre-calibrated.

In accordance with another embodiments, a method of analyzing a test sample is provided. The method is performed at a test sampling apparatus including (i) a housing extending from a first end to a second end opposite the first end, (ii) a chamber disposed at the first end of the housing, (iii) a test sensor fluidically coupled to the chamber and disposed within an interior portion of the housing, (iv) a cover configured to enclose the first end of the housing such that the chamber is covered, and (v) one or more processors disposed within an interior portion of the second end and communicatively coupled to the test sensor. The method includes receiving, via the chamber, a test sample from a user. The method further includes receiving, at the test sensor, a portion of the test sample, and generating, by the test sensor, information based on the portion of the test sample. The information includes data identifying one or more substances in the portion of the test sample. The method further includes receiving, at the one or more processors, the information from the test sensor, and analyzing the test sample based on the information. In some embodiments, the method includes displaying a test analysis.

In some embodiments, the test sampling apparatus further includes an absorbent material layer disposed over the chamber. The method further includes receiving, at the absorbent material layer, the test sample from the user, and storing the test sample at the absorbent material layer. The method includes dispersing, via the absorbent material layer, the test sample to the chamber when the cover is coupled to the first end of the housing. In some embodiments, the test sampling apparatus includes a buffer liquid stored within a foil seal used to calibrate the test sensor, and the method further includes piercing the foil seal upon first actuation of the cover.

In accordance with another embodiments, a method of fabricating a test sampling apparatus is provided. The method of fabricating the test sampling apparatus includes providing a housing that extends from a first end to a second end opposite the first end. The housing includes a chamber disposed at the first end; the chamber is configured to receive a test sample from a user. The method of fabricating the test sampling apparatus further includes providing a test sensor fluidically coupled to the chamber and disposed within an interior portion of the housing. The test sensor is configured to receive a portion of the test sample, and generate information based on the portion of the test sample. The information includes data corresponding to one or substances identified in the portion of the test sample. The method of fabricating the test sampling apparatus includes providing one or more processors disposed within an interior portion of the second end and communicatively coupled to the test sensor. The one or more processors are configured to receive the information from the test sensor and analyze the test sample based on the information. The method of fabricating the test sampling apparatus further includes providing a cover configured to enclose the first end of the housing such that the chamber is covered.

Note that the various embodiments described above can be combined with any other embodiments described herein. The features and advantages described in the specification are not all inclusive and, in particular, many additional features and advantages will be apparent to one of ordinary skill in the art in view of the drawings, specification, and claims.

### BRIEF DESCRIPTION OF THE DRAWINGS

So that the present disclosure can be understood in greater detail, a more particular description may be had by reference to the features of various embodiments, some of which are illustrated in the appended drawings. The appended drawings, however, merely illustrate pertinent features of the present disclosure and are therefore not to be considered limiting, for the description may admit to other effective features as the person of skill in this art will appreciate upon reading this disclosure.

FIG. 1 illustrates an example of a test sampling apparatus, in accordance with some embodiments.

FIGS. 2A and 2B illustrate operation use of a test sampling apparatus, in accordance with some embodiments.

FIGS. 3A and 3B illustrate a top view and a bottom view of a test sampling apparatus, in accordance with some embodiments.

FIGS. 4A and 4B illustrate exploded views of a test sampling apparatus, in accordance with some embodiments.

FIGS. 5A and 5B illustrate an alternate embodiment of a test sampling apparatus, in accordance with some embodiments.

FIGS. 6A and 6B illustrate an additional embodiment of a test sampling apparatus, in accordance with some embodiments.

FIGS. 7A and 7B illustrate an embodiment of a test sampling apparatus with a sliding cover, in accordance with some embodiments.

FIG. 8 is a flow diagrams illustrating a method of analyzing a test sample, in accordance with some embodiments.

In accordance with common practice, the various features illustrated in the drawings may not be drawn to scale. Accordingly, the dimensions of the various features may be arbitrarily expanded or reduced for clarity. In addition, some of the drawings may not depict all of the components of a given system, method, or device. Finally, like reference numerals may be used to denote like features throughout the specification and figures.

### DETAILED DESCRIPTION

Numerous details are described herein in order to provide a thorough understanding of the example embodiments illustrated in the accompanying drawings. However, some embodiments may be practiced without many of the specific details, and the scope of the claims is only limited by those features and aspects specifically recited in the claims. Furthermore, well-known processes, components, and materials have not been described in exhaustive detail so as to avoid obscuring pertinent aspects of the embodiments described herein.

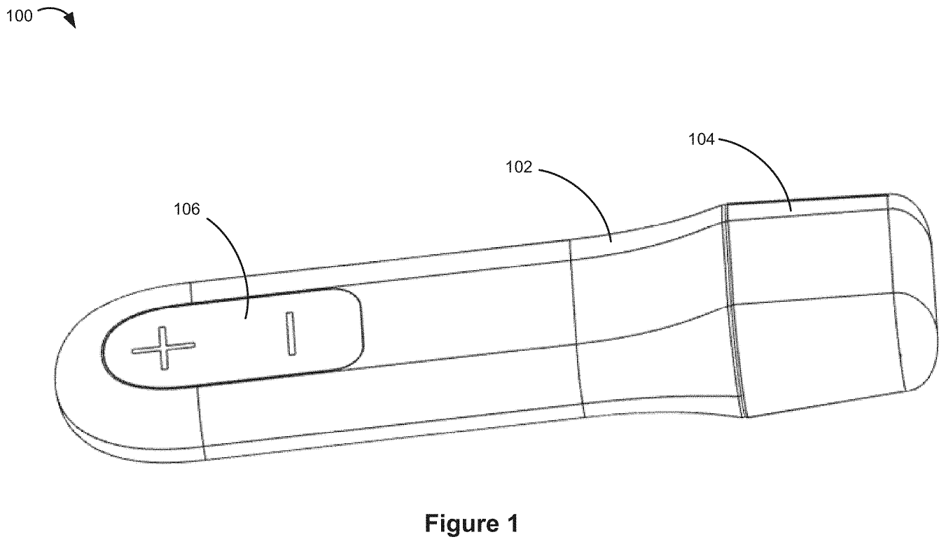

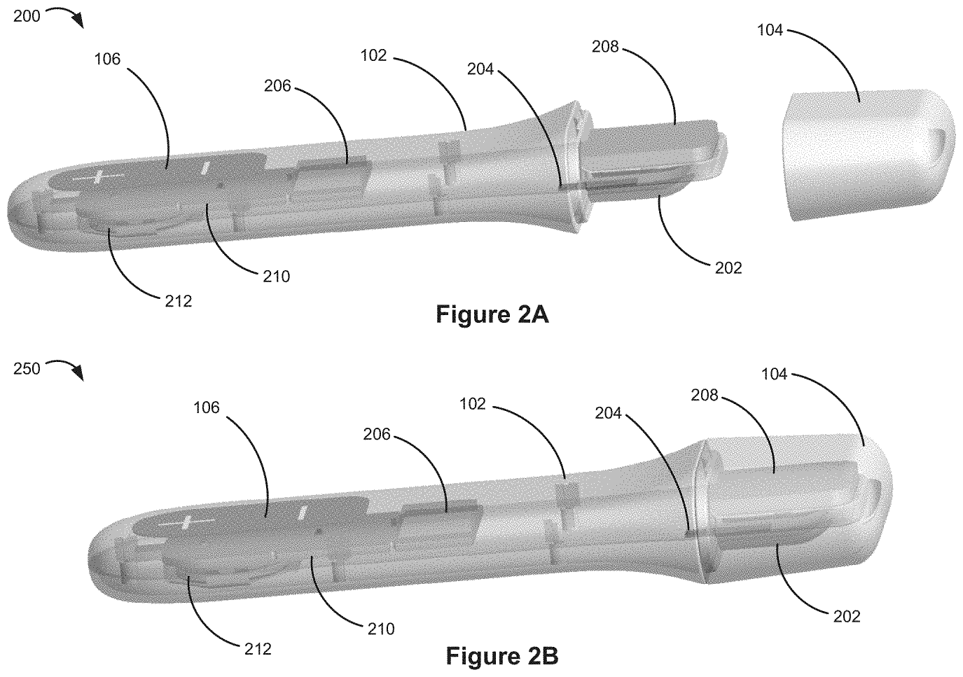

FIG. 1 illustrates an example of a test sampling apparatus, in accordance with some embodiments. The test sampling apparatus 100 is configured to receive and evaluate a biological test sample (e.g., urine). In some embodiments, the test sampling apparatus 100 includes a housing 102 extending from a first end to a second end opposite the first end. The housing 102 includes chamber 202, a test sensor 204, and/or one or more processors 206 (FIG. 2). The chamber 202, test sensor 204, and the one or more processors 206 are discussed in detail below in reference to FIG. 2. The test sampling apparatus 100 includes a cover 104 configured to enclose the first end of the housing 102 such that the chamber 202 is covered. In some embodiments, the test sampling apparatus 100 includes an interface 106 (such as a touch display, one or more buttons, light sources, etc.) disposed on an external portion of the housing 102. The interface 106 is configured to present a test analysis (determined by the test sensor 204 and the one or more processors 206 as discussed below in reference to FIGS. 2A and 2B) to the user.

FIGS. 2A and 2B illustrate operation use of a test sampling apparatus, in accordance with some embodiments. A first operational view 200 of the test sampling apparatus 100 (FIG. 1) shows the test sampling apparatus 100 with the cover 104 removed. A second operational view 250 shows the test sampling apparatus 100 with the cover 104 coupled to the housing 102. The test sampling apparatus 100 includes a housing 102, a chamber 202, a test sensor 204, one or more processors 206, a printed circuit board 210, a cover 104, and an interface 106. In some embodiments, the test sampling apparatus 100 further includes an absorbent material layer 208 disposed over the chamber 202. The test sampling apparatus 100 includes an internal power supply 212 configured to power the one or more of the processors 206, the test sensor 204, and the interface 106.

In the first operational view 200, the cover 104 is removed from the housing 102. The housing 102 is configured to house one or more components of the test sampling apparatus 100. In particular, the housing 102 houses at least the one or more processors 206 and the test sensor 204. In some embodiments, the housing further houses a portion of the chamber 202. In some embodiments, the chamber 104 disposed at the first end of the housing 102. When the cover 104 is not coupled to the housing 102, the portion of the hosing 102 that houses the chamber 202 is exposed to receive a biological test sample from a user, such as urine. The test sampling apparatus 100 analyzes the test sample as discussed below in reference to the test sensor 204 and the one or more processors 206.

The absorbent material layer 208 is configured to receive and store the test sample received from the user. In some embodiments, the absorbent material layer 208 is at least 1 mm thick. In some embodiments, the absorbent material layer 208 has a thickness between 1 to 3 mm (±0.2 mm). In some embodiments, the absorbent material layer 208 has a width of approximately 2.2 cm and a length of approximately 2.5 cm (where approximately is up to ±0.5 cm). In some embodiments, the absorbent material layer 208 is an absorbent polymer, cellulose filters, porous plastic, woven meshes, non-woven materials, gauze, cotton, natural sponges, etc. In some embodiments, the absorbent material layer 208 is configured to store at least 400 milliliters. The absorbent material layer 208 is further configured to disperse the test sample to the chamber 202 when compressed (as described below in reference to the second operational view 250).

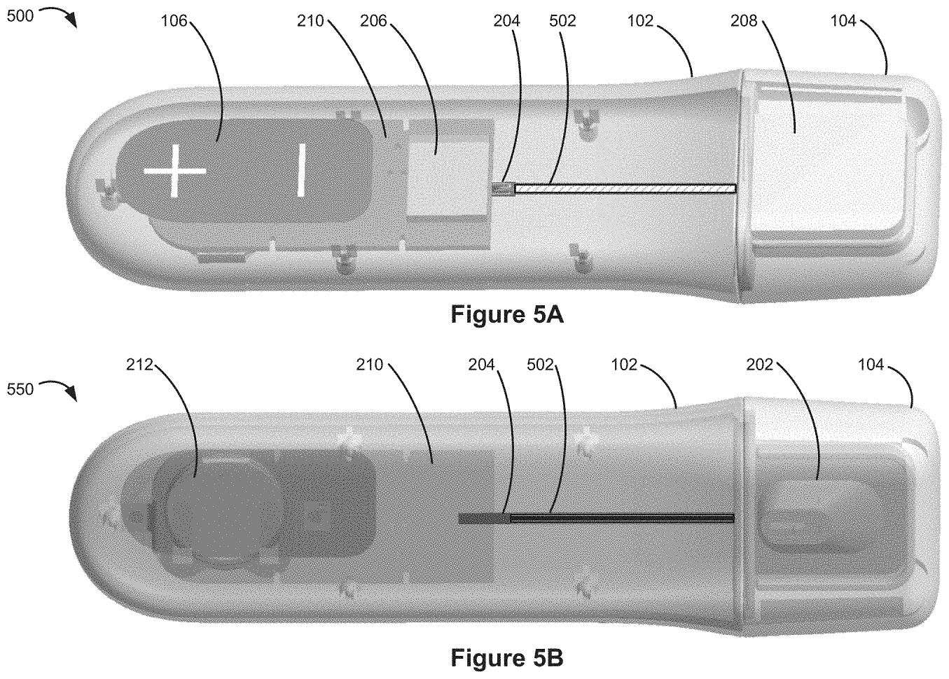

The chamber 202 receives a portion of the test sample from the absorbent material layer 208 (when the absorbent material layer 208 is compressed). The chamber 202 is partially cone or dome shaped such that fluid entering the chamber 202 flows downward (or to the base of the chamber 202). In some embodiments, the chamber 202 is configured to receive and/or hold approximately 400 μL (where approximately is up to ±50 μL). The chamber 202 is fluidically coupled to the test sensor 204 and configured to guide the received test sample to the test sensor 204. In some embodiments, the chamber 202 is coupled directly to or disposed over the test sensor 204. Alternatively, in some embodiments, the chamber 202 is indirectly coupled to the test sensor 204 as described below in reference to FIGS. 5A and 5B.

The test sensor 204 receives a portion of the test sample from the chamber 202. In some embodiments, the test sensor 204 is disposed within an interior portion of the housing 102. In some embodiments, the test sensor 204 is disposed at the first end of the housing 102. In some embodiments, the test sensors are pre-calibrated (e.g., calibrated during manufacturing). Alternatively, in some embodiments, a buffer liquid used to calibrate or provide baseline values for the test sensor. In some embodiments, the buffer liquid is stored within a foil seal (not shown) that is pierced when the cover 104 is coupled to the housing 102.

The test sensor 204, upon receiving the portion of the test sample from the chamber 202, generates information (based on the portion of the test sample) including data identifying one or more substances in the portion of the test sample. For example, the test sensor 204 may be configured to detect pathogens, metabolites, or physiological parameters of the test sample (e.g., pH). In some embodiments, the test sensor 204 is one of a FET-type device, a ChemFET-type device, EChemFET-type device, or electrochemical-type device. In some embodiments, the test sensor 204 is an immuno-assay. In some embodiments, isothermal DNA amplification can be employed to amplify the DNA and hence facilitate genetic screening for biomarkers. In some embodiments, the test sensor 204 can detect proteins or fragments of proteins. In some embodiments, the test sensor 204 can detect different forms of nucleic acid (including but not limited to DNA, mRNA, micro-RNA, siRNA). In some embodiments, the test sensor 204 can detect nucleic acid via amplification of specific nucleic acid sequence, or via detection of specific nucleic acid sequences, for instance through hybridization to an oligonucleotide or through a catalytically inactive CRISPR complex with a sgRNA having an oligonucleotide sequence that is complementary with a DNA sequence of interest.

Further details regarding the test sensor 204 and various detection methodologies that can be employed in connection with the test sampling apparatus 100 disclosed herein can be found in the following patents and published applications: U.S. Pat. No. 9,664,674, entitled “Device and Method for Chemical Analysis;” US Pat. Pub. No. 20 19/0079068, entitled “Device and Method for Chemical Analysis;” US Pat. Pub. No. 2019/0284615, entitled “Methods and Devices for Detection of Pathogens;” US Pat. Pub. No. 2020/00 11860, entitled “Functionalized Sensor for Detection of Biomarkers;” U.S. Pat. No. 10,782,285, entitled “Device and Method for Chemical Analysis;” and US Pat. Pub. No. 2020/03 00845, entitled “Methods and Devices for Detection of THC.” The entire contents of each of these publications is hereby incorporated by reference herein.

The test sensor 204 is in communication with the one or more processors 206. The test sensor 204 is configured to provide the one or more processors 206 the generated information. In some embodiments, the test sensor 204 is in communication with the one or more processors 206 via wireless or wired connection. For example, the test sensor 204 can be coupled to the one or more processors 206 via a ribbon cable, USB, or other connecting element. Alternatively, the test sensor 204 can be communicatively coupled to the one or more processors 206 via Bluetooth or other wireless protocol.

The one or more processors 206 are disposed within an interior portion of the housing 102. In some embodiments, the one or more processors 206 are disposed within the second end of the housing. In some embodiments, the one or more processors 206 are configured to analyze the generated information. The one or more processors 206 are configured to determine whether one or more target analytes of interest (e.g., one or more pathogens) are present in the test sample. In general, instructions for analyzing the generated information can be implemented in hardware, firmware, and/or software using techniques known in the art as informed by the present teachings. In some embodiments, the one or more instructions are stored in a computer memory or computer-readable storage medium coupled to the one or more processors.

In some embodiments, the one or more processors 206 are coupled to the printed circuit board 210 on which electronic circuitry is disposed. The one or more processors 206 and the electric circuitry disposed on the printed circuit board 210 are powered by the internal power source 212 (e.g., batteries). In some embodiments, the one or more processors 206 are communicatively coupled to the interface 106. In some embodiments, the interface 106 is powered by the internal power source 212.

The interface 106 includes one or more of a display, one or more buttons, and/or one or more light sources (e.g., light emitting diodes). In some embodiments, the interface 106 receives, from the one or more processors 206, a determined test analysis and presents the determined test analysis to the user. In some embodiments, the interface 106 illuminates a positive or negative sign using a light emitting diode or other illumination source to indicate a positive or negative test result. In other embodiments, the interface 106 displays a numeric or alphanumeric result indication. In some embodiments, the interface 106 receives one or more inputs from a user, such as actuation of one or more buttons (e.g., surfaces that can be depressed or touch surfaces that can be selected by a user to turn the device on, off, initiate a test, etc.). In some embodiments, the test sampling apparatus 100 can be coupled to a remote digital data processor, e.g., a smart phone, tablet, computer, network, etc., and can communicate results and/or receive inputs via a wired or wireless connection to the remote digital data processor. For example, in one embodiment the test sampling apparatus 100 can be coupled to a smart phone or other external device via a Bluetooth wireless connection using a wireless communication interface included in the device. Test results can be communicated to, and displayed by, the remote digital data processor.

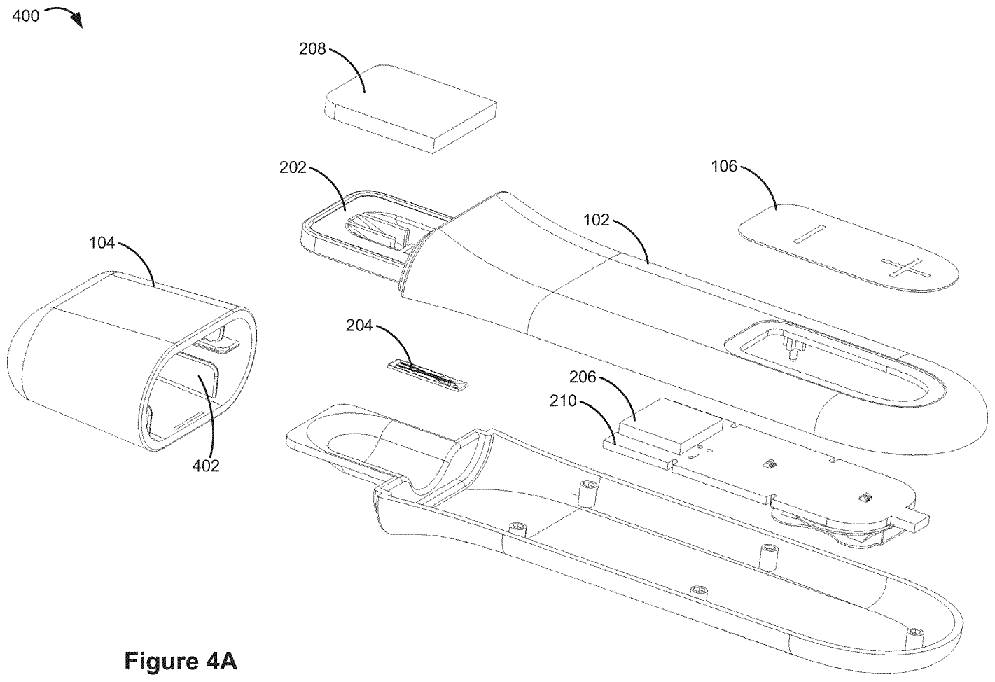

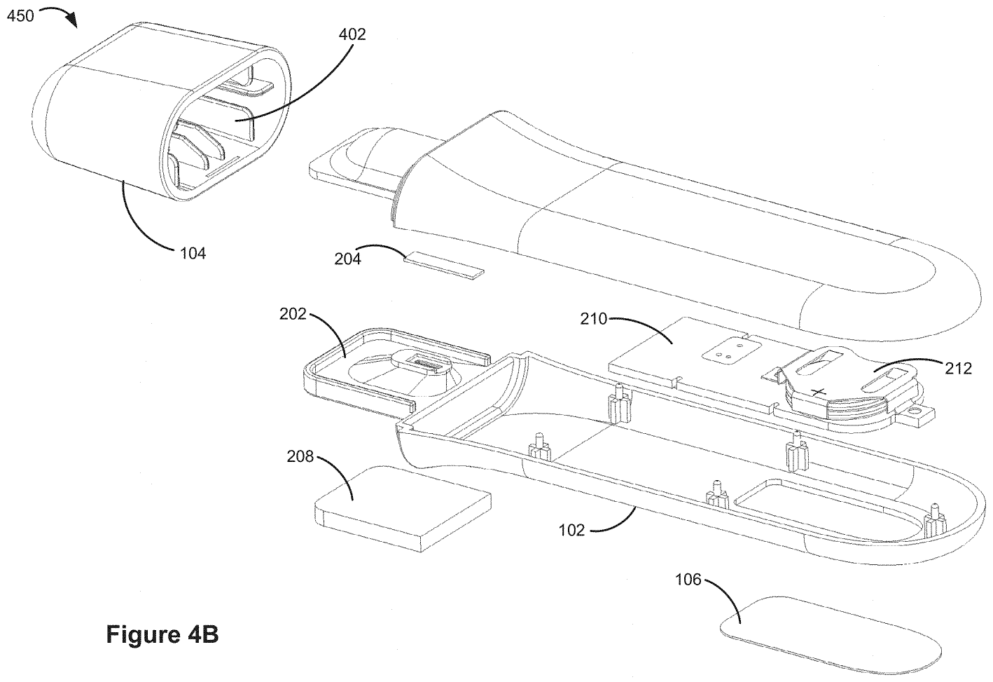

In the second operational view 250, the cover 104 is coupled to the housing 102. In some embodiments, when the cover 104 is coupled to the first end of the housing 102, the absorbent material layer 208 is compressed such that a portion of the stored or absorbed test sample is dispersed from the absorbent material layer 208. As described above, the dispersed test sample is provided to the chamber 202 for testing by the test sensor 104. The cover 104 provides a clean and easy way to extract the test sample from the absorbent material layer 208. Additionally, the cover 104 allows the user to prevent excess test sample from leaking or escaping from the test sampling apparatus 100. In some embodiments, the cover 104 is a cap. Additional information on the cap is provided below in reference to FIGS. 4A and 4B.

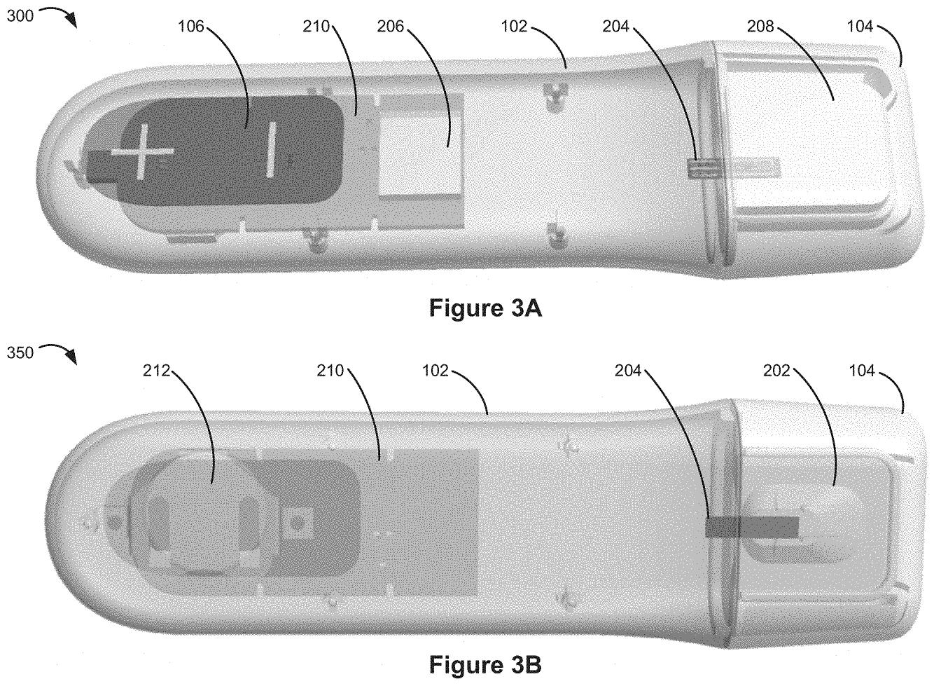

FIGS. 3A and 3B illustrate a top view and a bottom view of a test sampling apparatus, in accordance with some embodiments. The top view 300 of the test sampling apparatus 100 (FIG. 1) shows a housing 102, a cover 104, a test sensor 204, one or more processors 206, a printed circuit board 210, an absorbent material layer 208, and an interface 106. The bottom view 350 of the test sampling apparatus 100 shows the housing 102, the cover 104, the test sensor 204, the chamber 202, the printed circuit board 210, and an internal power source 212.

FIGS. 4A and 4B illustrate exploded views of a test sampling apparatus, in accordance with some embodiments. A first exploded view 400 shows a top view of the test sampling apparatus 100 (FIG. 1), and the second exploded view 450 shows a bottom view of the test sampling apparatus. The test sampling apparatus in the first exploded view 400 and the second exploded view 450 includes a housing 102, a cover 104, a chamber, a test sensor 204, one or more processors 206, an absorbent material layer 208, a printed circuit board 210, an internal power source 212, and an interface 106. As described above, in some embodiments, the cover 104 is a cap. In some embodiments, the cap includes one or more ribs 402. In some embodiments, the one or more ribs 402 are configured to further compress the absorbent material layer 208 such that additional fluid is dispersed from the absorbent material layer 208. In some embodiments, the cap has a width of 41 mm (±0.5 mm), a height of 21 mm (±0.5 mm), and a length of 32 mm (±0.5 mm).

FIGS. 5A and 5B illustrate an alternate embodiment of a test sampling apparatus, in accordance with some embodiments. A first view 500 shows a top view of the alternate test sampling apparatus, and a second view 550 shows a bottom view of the alternate test sampling apparatus. The alternate test sampling apparatus includes similar features to the test sampling apparatus discussed above in reference to FIGS. 1-4B. For example, the alternate test sampling apparatus may include a housing 102, a cover 104, a chamber 202, a test sensor 204, one or more processors 206, an absorbent material layer 208, a printed circuit board 210, an internal power source 212, and an interface 106 as described above in reference to FIGS. 1-4B. The alternate test sampling apparatus further includes a channel 502 that fluidically couples the test sensor 204 to the chamber 202.

In some embodiments, the test sensor 204 is disposed at the second end of the housing 102, remote from the chamber 202. In some embodiments, the test sensor 204 is electrically coupled to the printed circuit board 210. Alternatively, in some embodiments, the test sensor 204 is integral with the one or more processors 206. In some embodiments, the alternate embodiment of the test sampling apparatus includes a channel 502 that transfers a test sample received from the user at the chamber 202 to the test sensor 204.

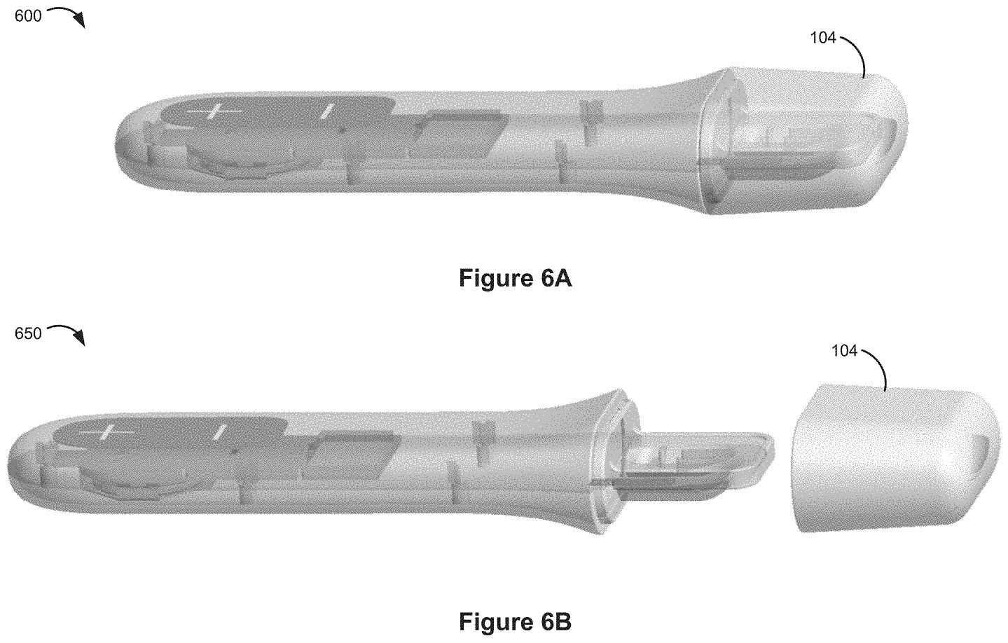

FIGS. 6A and 6B illustrate an additional embodiment of a test sampling apparatus, in accordance with some embodiments. A first view 600 shows a cover 104 coupled to the additional test sampling apparatus and a second view 650 shows the cover 104 decoupled from the additional test sampling apparatus. The alternate test sampling apparatus includes similar features to the test sampling apparatus discussed above in reference to FIGS. 1-4B. For example, the additional test sampling apparatus may include a housing 102, a cover 104, a chamber 202, a test sensor 204, one or more processors 206, a printed circuit board 210, an internal power source 212, and an interface 106 as described above in reference to FIGS. 1-4B. The additional embodiment of the test sampling apparatus shows an embodiment without the absorbent material layer 208 (FIG. 2). In some embodiments, the test sample is provided directly to the chamber 202, which in turns provides a portion of the test sample to the test sensor 204.

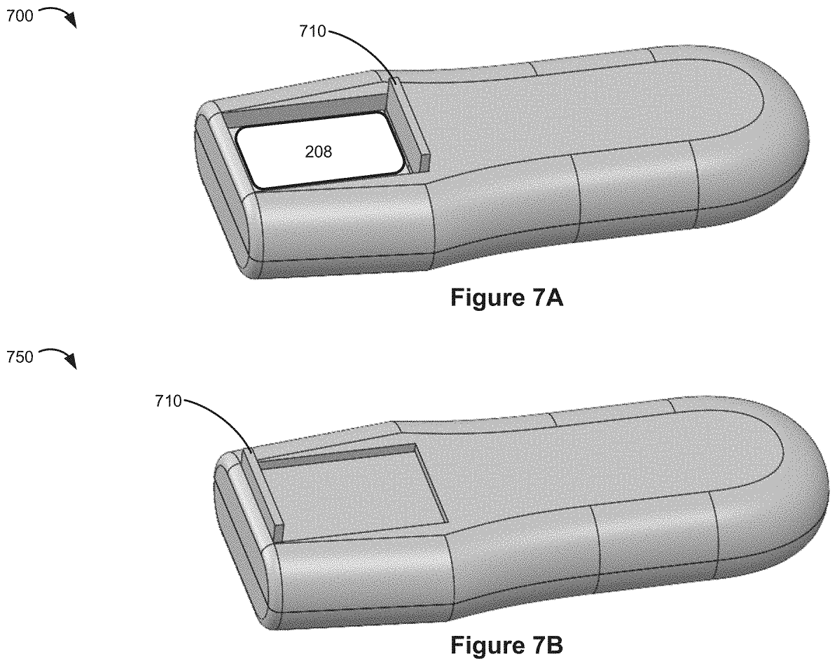

FIGS. 7A and 7B illustrate an embodiment of a sliding cover test sampling apparatus, in accordance with some embodiments. A first view 700 of the sliding cover test sampling apparatus shows a sliding cover 710 in an open position and a second view 750 of the sliding cover test sampling apparatus shows the sliding cover 710 in a closed position. The alternate test sampling apparatus includes similar features to the test sampling apparatus discussed above in reference to FIGS. 1-4B. For example, the sliding cover test sampling apparatus may include a housing 102, a cover 104, a chamber 202, a test sensor 204, one or more processors 206, an absorbent material layer 208, a printed circuit board 210, an internal power source 212, and an interface 106 as described above in reference to FIGS. 1-4B. In some embodiments, when the sliding cover 710 is in the open position (as shown in the first view 700), the sliding cover test sampling apparatus is configured to receive a test sample from a user for analysis. In some embodiments, the sliding cover test sampling apparatus includes an absorbent material layer 208 such that the test sample provided by the user is absorbed and held by the absorbent material layer 208. In some embodiments, when the sliding cover 710 is moved to the closed position (as shown in the second view 750), the sliding cover test sampling apparatus is configured to compress the absorbent material layer 208 (e.g., via the sliding cover 710) such that a portion of the stored or absorbed test sample is dispersed from the absorbent material layer 208 to a chamber 202 and a test sensor 204 of the sliding cover test sampling apparatus for analysis. In some embodiments, the sliding cover test sampling apparatus does not include the absorbent material layer 208, such that the test sample is provided directly to the chamber 202, which in turns provides a portion of the test sample to the test sensor 204. The sliding cover 710 is configured to prevent the test sample from leaking or escaping from the sliding cover test sampling apparatus.

The above examples of the test sampling apparatus are non-limiting and simplified for ease of explanation.

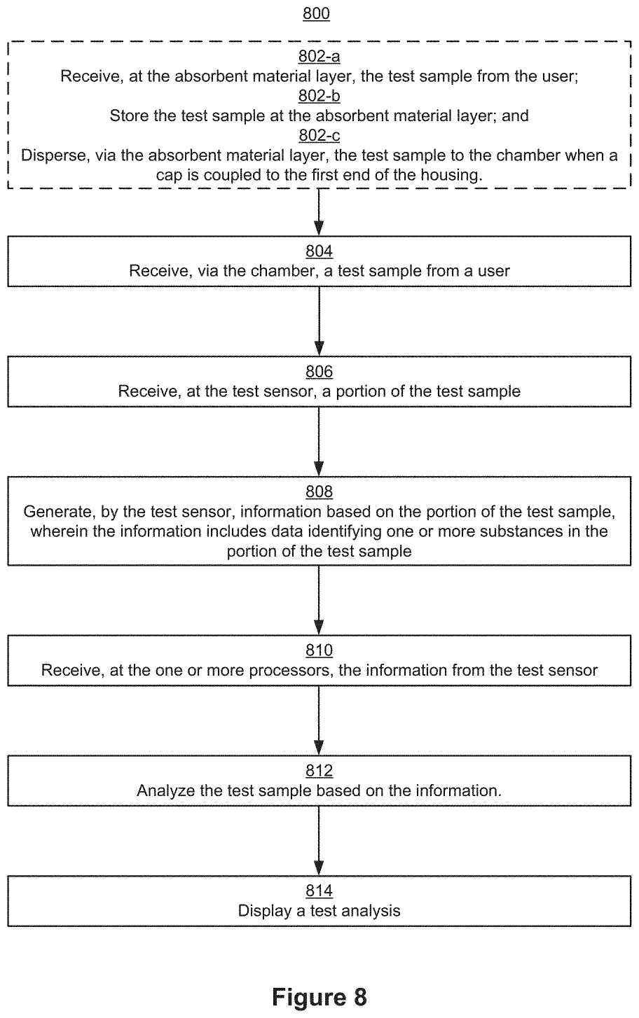

FIG. 8 is a flow diagrams illustrating a method 800 of analyzing a test sample, in accordance with some embodiments. Operations (e.g., steps) of the method 800 may be performed by one or more processors 205 (FIG. 2) of a test sampling apparatus 100 (FIG. 1). At least some of the operations shown in FIG. 8 correspond to instructions stored in a computer memory or computer-readable storage medium. Operations 802-814 can also be performed in part using one or more processors and/or using instructions stored in memory or computer-readable medium of a computing device (such as a smart phone, tablet, computer, etc. that can perform operations 802-814 alone or in conjunction with the one or more processors of the test sampling apparatus 100).

In some embodiments, the method 800 includes receiving (802-a), at an absorbent material layer, a test sample from the user. The method 800 also includes storing (802-b) the test sample at the absorbent material layer stores, and dispersing (802-c), via the absorbent material layer, the test sample to the chamber when a cap is coupled to the first end of the housing. The method 800 includes receiving (804), via the chamber, the test sample from the user.

The method 800 includes receiving (806), at the test sensor, a portion of the test sample and generating (808), by the test sensor, information based on the portion of the test sample. In some embodiments, the information includes data identifying one or more substances in the portion of the test sample. The method 800 further includes receiving (810), at the one or more processors, the information from the test sensor, and analyzing (812) the test sample based on the information. In some embodiments, the method further includes displaying (814) a test analysis to the user.

It will be understood that, although the terms “first,” “second,” etc. may be used herein to describe various elements, these elements should not be limited by these terms. These terms are only used to distinguish one element from another.

The terminology used herein is for the purpose of describing particular embodiments only and is not intended to be limiting of the claims. As used in the description of the embodiments and the appended claims, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” can be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” can be construed to mean “upon determining” or “in response to determining” or “in accordance with a determination” or “upon detecting” or “in response to detecting” that the stated condition precedent is true, depending on the context.

The foregoing description, for purpose of explanation, has been described with reference to specific embodiments. However, the illustrative discussions above are not intended to be exhaustive or to limit the claims to the precise forms disclosed. Many modifications and variations are possible in view of the above teachings. The embodiments were chosen and described in order to best explain principles of operation and practical applications, to thereby enable others skilled in the art.

What is claimed is:

1. A test sampling apparatus comprising:
a housing extending from a first end to a second end opposite the first end, the housing comprising a chamber disposed at the first end, the chamber configured to receive a test sample from a user;
a test sensor fluidically coupled to the chamber and disposed within an interior portion of the housing, wherein the test sensor is configured to receive a portion of the test sample and generate information based thereon, wherein the information includes data identifying one or more substances in the portion of the test sample;
one or more processors disposed within an interior portion of the second end and in communication with the test sensor, the one or more processors configured to receive the information from the test sensor and analyze the test sample based on the information; and
a cover configured to enclose the first end of the housing such that the chamber is covered.

2. The test sampling apparatus of claim 1, further comprising a display disposed on an external portion of the housing and responsive to the one or more processors, the display configured to present a test analysis.

3. The test sampling apparatus of claim 1, wherein the cover is a cap configured to couple to the first end of the housing.

4. The test sampling apparatus of claim 3, further comprising an absorbent material layer disposed over the chamber, wherein the absorbent material layer is configured to:
receive the test sample received from the user;
store the test sample; and
disperse the test sample to the chamber when the cap is coupled to the first end of the housing.

5. The test sampling apparatus of claim 4, wherein the cap includes a plurality of ribs for compressing the absorbent material layer.

6. The test sampling apparatus of claim 1, wherein the cover is configured to slidably retract to expose the chamber.

7. The test sampling apparatus of claim 1, wherein the test sensor is one of a ChemFET-type device, EChemFET-type device, immuno-assay, or electrochemical-type device.

8. The test sampling apparatus of claim 1, wherein the test sensor is configured to detect proteins or fragments of proteins.

9. The test sampling apparatus of claim 1, wherein the test sensor is configured to detect nucleic acid via amplification of specific nucleic acid sequence, via detection of specific nucleic acid sequences.

10. The test sampling apparatus of claim 1, the test sensor is configured to perform isothermal DNA amplification that is used for detecting the signals based on the test sample.

11. The test sampling apparatus of claim 1, wherein the test sensor is disposed at the first end of the housing.

12. The test sampling apparatus of claim 1, wherein the test sensor is disposed at the second end of the housing, and the test sampling apparatus further comprises a channel for transferring the test sample from the chamber to the test sensor.

13. The test sampling apparatus of claim 12, wherein the test sensor is integral with the one or more processors.

14. The test sampling apparatus of claim 1, further comprising a buffer liquid used to calibrate the test sensor.

15. The test sampling apparatus of claim 14, wherein the buffer liquid is stored within a foil seal, and the cover is configured to pierce the foil seal upon first actuation.

16. The test sampling apparatus of claim 1, wherein the test sensor is pre-calibrated.

17. The test sampling apparatus of claim 1, wherein the test sample is urine.

18. A method of analyzing a test sample, the method comprising:
at a test sampling apparatus including (i) a housing extending from a first end to a second end opposite the first end, (ii) a chamber disposed at the first end of the housing, (iii) a test sensor fluidically coupled to the chamber and disposed within an interior portion of the housing, (iv) a cover configured to enclose the first end of the housing such that the chamber is covered, and (v) one or more processors disposed within an interior portion of the second end and communicatively coupled to the test sensor:
receiving, via the chamber, a test sample from a user;
receiving, at the test sensor, a portion of the test sample;
generating, by the test sensor, information based on the portion of the test sample, wherein the information includes data identifying one or more substances in the portion of the test sample;
receiving, at the one or more processors, the information from the test sensor; and
analyzing the test sample based on the information.

19. The method of claim 18, wherein the test sampling apparatus further includes an absorbent material layer disposed over the chamber, the method further comprising:
receiving, at the absorbent material layer, the test sample from the user;
storing the test sample at the absorbent material layer; and
dispersing, via the absorbent material layer, the test sample to the chamber when the cover is coupled to the first end of the housing.

20. The method of claim 18, wherein the test sampling apparatus includes a buffer liquid stored within a foil seal used to calibrate the test sensor, and the method further comprises piercing the foil seal upon first actuation of the cover.
